(19) United States
(12) Patent Application Publication
(10) Pub. No.: US 2024/0206522 A1
(43) Pub. Date: Jun. 27, 2024

(54) Nicotine Pouch Assembly

(71) Applicants: Jeffery Dionne, Laurel, MT (US); Dylan Clouse, Columbus, MT (US)

(72) Inventors: Jeffery Dionne, Laurel, MT (US); Dylan Clouse, Columbus, MT (US)

(21) Appl. No.: 18/086,806

(22) Filed: Dec. 22, 2022

(51) Int. Cl. A24B 15/16; A24B 13/00; A24F 23/00; A61K 9/00; A61K 31/465; B65D 51/24; B65D 85/07

(52) U.S. Cl. CPC A24B 15/16; A24B 13/00; A24F 23/00; A61K 9/009; A61K 31/465; B65D 51/245; B65D 85/07

(57) ABSTRACT

A nicotine pouch assembly includes a canister that is comprised of a fluid impermeable material. A lid is removably attachable to the canister for opening and closing the canister. A plurality of nicotine packets is each stored within the canister. Each of the nicotine packets is filled with a non-plant material and the non-plant material in each of the nicotine packets is infused with nicotine. A selected one of nicotine packets is insertable into a user's mouth such that the selected nicotine packet 22 is exposed to the user's saliva when the selected nicotine packet 22 is positioned in the user's mouth. In this way the nicotine packet in the user's mouth can release the nicotine into the user's saliva.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to nicotine devices and more particularly pertains to a new nicotine device for facilitating a user to enjoy nicotine without the use of tobacco. The device includes a canister, a lid removably attachable to the canister and plurality of nicotine packets contained in the canister. Each of the nicotine packets contains a granular salt that is infused with nicotine.

### (2) Description of Related Art Including Information Disclosed Under 37 CFR 1.97 and 1.98

The prior art relates to nicotine devices including a nicotine pouch that includes a nicotine barrier layer and nicotine degradation agent barrier. The prior art discloses a nicotine pouch that includes a powder of at least one free nicotine salt, at least one pH adjusting agent and at least one filler. The prior art discloses a method of producing an oral pouched snuff product that comprises a filling material which has a moisture content of at least 20%. The prior art discloses a nicotine-containing pharmaceutical product that includes a water permeable pouch, a nicotine containing pharmaceutical product and product identifying information. The prior art discloses an oral nicotine pouch which is filled with tobacco and which has a length ranging between 3.0 mm and 10.0 mm. The prior art discloses a nicotine pouch that includes a packaging material which comprises at least 50% cellulose staple fibers and no more than 50% thermoplastic fibers.

### BRIEF SUMMARY OF THE INVENTION

An embodiment of the disclosure meets the needs presented above by generally comprising a canister that is comprised of a fluid impermeable material. A lid is removably attachable to the canister for opening and closing the canister. A plurality of nicotine packets is each stored within the canister. Each of the nicotine packets is filled with a non-plant material and the non-plant material in each of the nicotine packets is infused with nicotine. A selected one of nicotine packets is insertable into a user's mouth such that the selected nicotine packet 22 is exposed to the user's saliva when the selected nicotine packet 22 is positioned in the user's mouth. In this way the nicotine packet in the user's mouth can release the nicotine into the user's saliva.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is an exploded perspective view of a nicotine pouch assembly according to an embodiment of the disclosure.

FIG. 2 is a perspective view of a nicotine pouch of an embodiment of the disclosure.

FIG. 3 is a top view of a nicotine pouch of an embodiment of the disclosure.

FIG. 4 is a front view of a nicotine pouch of an embodiment of the disclosure.

FIG. 5 is a cross sectional view taken along line 5-5 of FIG. 3 of an embodiment of the disclosure.

FIG. 6 is a perspective in-use view of an embodiment of the disclosure.

### DETAILED DESCRIPTION OF THE INVENTION

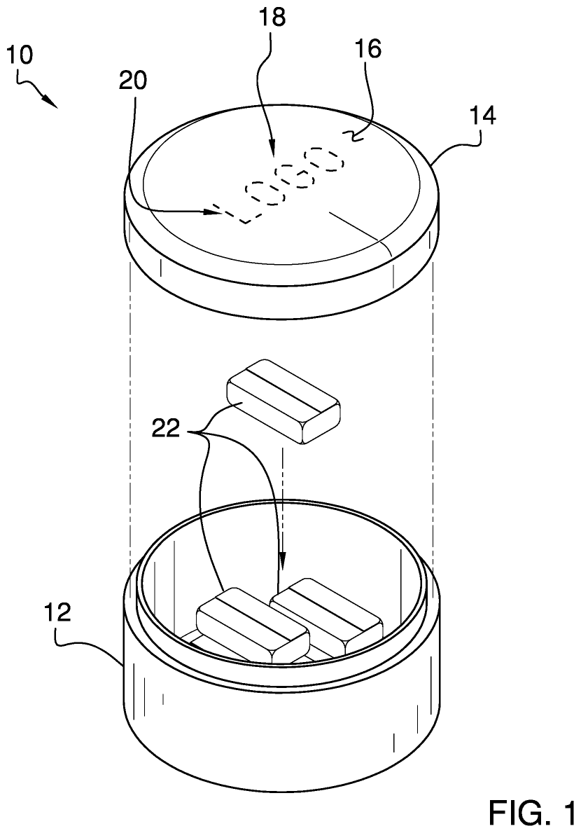

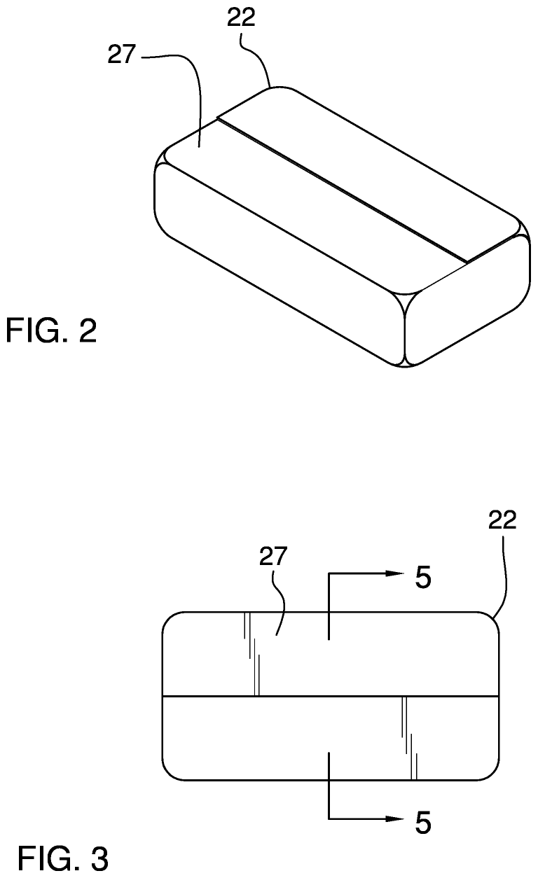

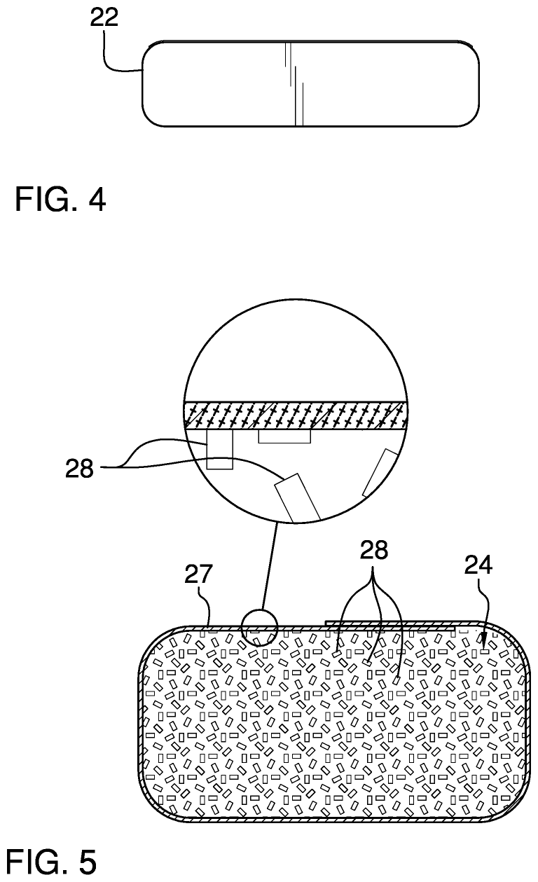

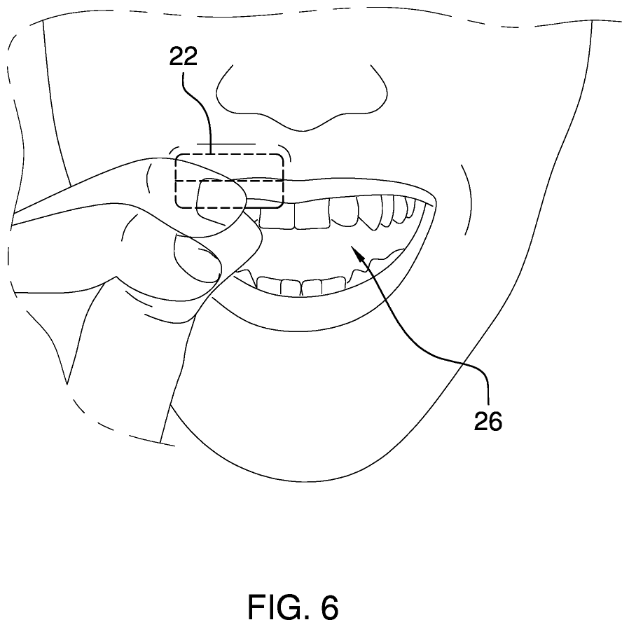

With reference now to the drawings, and in particular to FIGS. 1 through 6 thereof, a new nicotine device embodying the principles and concepts of an embodiment of the disclosure and generally designated by the reference numeral 10 will be described.

As best illustrated in FIGS. 1 through 6, the nicotine pouch assembly 10 generally comprises a canister 12 that is comprised of a fluid impermeable material. A lid 14 is removably attachable to the canister 12 for opening and closing the canister 12. The lid 14 has a top surface 16, indicia 18 are applied to the top surface 16 and the indicia 18 comprise an image of a logo 20. The logo 20 may be a logo of a manufacturer or any other visually appealing logo for marketing and branding purposes. Furthermore, the canister 12 and the lid 14 may have combined dimensions that are sufficiently small to facilitate the canister 12 and the lid 14 to be carried in clothing pockets.

A plurality of nicotine packets 22 is provided and each of the plurality of nicotine packets 22 is stored within the canister 12. Each of the nicotine packets 22 is filled with a non-plant material 24 and the non-plant material 24 in each of the nicotine packets 22 is infused with nicotine. A selected one of nicotine packets 22 is insertable into a user's mouth 26 such that the selected nicotine packet 22 is exposed to the user's saliva when the selected nicotine packet 22 is positioned in the user's mouth 26. In this way the nicotine packet 22 in the user's mouth 26 releases the nicotine into the user's saliva. As is most clearly shown in FIG. 5, the nicotine packets 22 are folded over onto themselves on a top side 27 of the nicotine packets 22.

Each of the nicotine packets 22 is comprised of a fluid permeable material, such as paper or other type of porous, organic material. In this way the plurality of nicotine packets 22 facilitate the user's saliva to penetrate the nicotine packets 22. The non-plant material in each of the nicotine packets 22 comprises a granular salt 28 that is infused with nicotine thereby facilitating the granular salt 28 to dissolve in a liquid. In this way the nicotine packet 22 in the user's mouth 26 can release the nicotine into the user's saliva. The granular salt 28 in each of the plurality of nicotine packets 22 is infused with a chemical flavor and the chemical flavor is released into the user's saliva for enhancing the user's pleasure. The chemical flavor may be a tobacco flavor, a whiskey barrel flavor or a tasteless flavor. Each of the nicotine packets 22 has a length of at least 38.0 mm and a width of at least 19.0 mm and a thickness of at least 9.5 mm. In this way each of the nicotine packets 22 facilitates the user to enjoy the physical sensation of having the selected nicotine packet 22 in the user's mouth 26.

In use, the user removes one or more nicotine packets 22, depending on the user's preference, from the canister 12 and the one or more nicotine packets 22 are positioned in the user's mouth 26. In this way the user's saliva penetrates the one or more nicotine packets 22 and dissolves the granular salt 28 in the nicotine packets 22. Thus, the nicotine that is infused into the granular salt 28 is released into the user's saliva thereby facilitating the user to enjoy the chemical effects of the nicotine.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A nicotine pouch assembly for facilitating a plurality of nicotine packets to be positioned in a porous pouch that is insertable into a user's mouth, said assembly comprising:
a canister being comprised of a fluid impermeable material;
a lid being removably attachable to said canister for opening and closing said canister, said lid having a top surface, said lid having indicia being applied to said top surface, said indicia comprising an image of a logo; and
a plurality of nicotine packets, each of said plurality of nicotine packets being stored within said canister, each of said nicotine packets being filled with a non-plant material, said non-plant material in each of said nicotine packets being infused with nicotine, a selected one of nicotine packets being insertable into a user's mouth wherein said selected nicotine packet 22 is configured to be exposed to the user's saliva when said selected nicotine packet 22 is positioned in the user's mouth thereby facilitating said nicotine packet in the user's mouth to release said nicotine into the user's saliva.

2. The assembly according to claim 1, wherein:
each of said nicotine packets is comprised of a fluid permeable material wherein each of said plurality of nicotine packets is configured to facilitate the user's saliva to penetrate said nicotine packets; and
each of said nicotine packets contains a granular salt being infused with nicotine thereby facilitating said granular salt to dissolve in a liquid wherein said nicotine packet in the user's mouth is configured to release the nicotine into the user's saliva.

3. The assembly according to claim 2, wherein said granular salt in each of said plurality of nicotine packets is infused with a chemical flavor wherein said nicotine packet in the user's mouth is configured to release the chemical flavor into the user's saliva for enhancing the user's pleasure.

4. The assembly according to claim 1, wherein each of said nicotine packets has a length of at least 38.0 mm and a width of at least 19.0 mm and a thickness of at least 9.5 mm wherein each of said nicotine packets is configured to facilitate the user to enjoy the physical sensation of having the selected nicotine pouch in the user's mouth.

5. A nicotine pouch assembly for facilitating a plurality of nicotine packets to be positioned in a porous pouch that is insertable into a user's mouth, said assembly comprising:
a canister being comprised of a fluid impermeable material;
a lid being removably attachable to said canister for opening and closing said canister, said lid having a top surface, said lid having indicia being applied to said top surface, said indicia comprising an image of a logo; and
a plurality of nicotine packets, each of said plurality of nicotine packets being stored within said canister, each of said nicotine packets being filled with a non-plant material, said non-plant material in each of said nicotine packets being infused with nicotine, a selected one of nicotine packets being insertable into a user's mouth wherein said selected nicotine packet 22 is configured to be exposed to the user's saliva when said selected nicotine packet 22 is positioned in the user's mouth thereby facilitating said nicotine packet in the user's mouth to release said nicotine into the user's saliva, each of said nicotine packets being comprised of a fluid permeable material wherein each of said plurality of nicotine packets is configured to facilitate the user's saliva to penetrate said nicotine packets, each of said nicotine packets containing a granular salt being infused with nicotine thereby facilitating said granular salt to dissolve in a liquid wherein said nicotine packet in the user's mouth is configured to release the nicotine into the user's saliva, said granular salt in each of said plurality of nicotine packets being infused with a chemical flavor wherein said nicotine packet in the user's mouth is configured to release the chemical flavor into the user's saliva for enhancing the user's pleasure, each of said nicotine packets having a length of at least 38.0 mm and a width of at least 19.0 mm and a thickness of at least 9.5 mm wherein each of said nicotine packets is configured to facilitate the user to enjoy the physical sensation of having the selected nicotine pouch in the user's mouth.

6. A method of improving the experience of using a nicotine pouch, the steps of the method comprising:
providing a canister;
providing a lid being removably attachable to said canister;
providing a plurality of nicotine packets, each of said plurality of nicotine packets containing a granular salt being infused with each of nicotine and a chemical flavor, each of said nicotine packets having a length and a width being sufficiently large to enhance the physical sensation of placing one of said nicotine packets in a user's mouth, each of said nicotine packets being comprised of a fluid permeable material thereby facilitating said granular salt in said nicotine packets to dissolve when said nicotine packets are exposed to liquid;
storing each of said nicotine packets in said canister;
removing a selected one of said nicotine packets from said canister; and
placing said selected nicotine pouch into the user's mouth thereby facilitating said nicotine in said granular salt to be dissolved in the user's saliva.
